(19) United States
(12) Patent Application Publication
(10) Pub. No.: US 2004/0159561 A1
(43) Pub. Date: Aug. 19, 2004

(54) Storage device for medical swabs

(76) Inventor: Andreas Fellinger, Ailertchen (DE)

Correspondence Address: D. PETER HOCHBERG CO. L.P.A., 1940 EAST 6TH STREET, CLEVELAND, OH, 44114, US

(21) Appl. No.: 10/772,633

(22) Filed: Feb. 5, 2004

Related U.S. Application Data
(62) Division of application No. 09/308,408, filed on Jun. 28, 1999.
Application No. 09/308,408 is a 371 of international application No. PCT/EP97/05609, filed on Oct. 10, 1997.

(30) Foreign Application Priority Data
Nov. 27, 1996 (DE) 296 20 636.9

(51) Int. Cl. A61B 17/06

(52) U.S. Cl. 206/63.3

(57) ABSTRACT

A storage device for medical swabs having an envelope for the latter is characterized in that the envelope is formed by adjacently disposed chambers which are sealed bacteria-tight for the swabs. The size of the chambers is determined by the number and the size of the swabs kept in stock in each of them, it being intended, so ensure absolute sterility, that only one swab should be kept in stock in one chamber, so that each swab can be removed sterilized from the storage device. The envelopes can be kept in stock rolled up or concertinaed.

### CROSS REFERENCE TO RELATED APPLICATIONS

This application is a divisional application of Application No. 09/308,408, filed Jun. 28, 1999, which is the National Stage of International Application No. PCT/EP97/05609 filed Sep. 10, 1997.

### BACKGROUND OF THE INVENTION

1. Field of the Invention

The invention relates to a storage device for swabs, in particular, sterilized medical swabs.

2. Description of Prior Art

Medical swabs are kept in stock sterilized in a multipack. They are used singly and are taken singly from a multipack. After opening of a multipack and removal of the first swab or swabs, the sterility of the remaining swabs in stock is no longer ensured so that they have to be resterilized. It is not possible to ensure sterility of the swabs present in a multipack once it has been opened even if the multipack is disposed in another, secondary pack.

### SUMMARY OF THE INVENTION

The invention is based on the object of producing a storage device which allows sterilized swabs to be removed one at a time or singly, while ensuring the sterility of the remaining swabs in stock in the storage device. It is an additional intention to minimize the space required for the storage device.

This object is achieved in a storage device characterized in that the envelope is formed by adjacently disposed chambers, which are sealed bacteria-tight, for the swabs, the size of the chambers being determined by the number and the size of the swabs kept in stock in each of these, and in that the envelope can be kept in stock rolled up or concertinaed.

The storage device according to the invention has an envelope consisting of single chambers for the swabs; these chambers are sealed bacteria-tight, and can be disposed adjacently. The size of the chambers is determined by the number and the size of the swabs kept in stock in each of them, it being intended, so ensure absolute sterility, that only one swab should be kept in stock in one chamber, so that each swab can be removed sterilized from the storage device. For areas of medicine where a large number of swabs is used, the sterility of a second swab which is kept in stock and, after removal of the first swab, remains for a short time in the same chamber, is likewise ensured so that it is also perfectly possible for two swabs to be kept in stock in one chamber in a storage device for swabs used in such areas. The envelope must be sufficiently flexible so that it can be kept in stock rolled up or concertinaed.

To this end, the envelope can consist of a flexible base layer and of a flexible covering layer bonded thereto, and one of these layers has areas of convexity relative to the other layer to form the chambers.

It is possible to provide strips intended to be torn through between the chambers in order to facilitate removal of a swab from the storage device and in order to improve overall the convenience of using the latter.

The chambers can be provided in a row or in several parallel rows and, in order to save space, the chambers in adjacent rows can be disposed offset relative to one another in order to minimize the spaces between.

The chambers can be formed in a simple manner by receiving depressions in the base layers which are covered by the covering layer. It is also possible, moreover, for each receiving depression to be sealed by an isolated covering layer.

The receiving depressions can moreover be produced by a thermoforming process, and the disposition of the covering layer or of the individual covering layers can take place by welding or sealing in order to produce integral bonding between the layers.

To facilitate opening of the chambers, they can be provided with aids to opening known to a skilled person. The convenience of use of the storage device according to the invention can be considerably improved by it being disposed rolled up or concertinaed in a dispenser in the form of a secondary pack which has a removal opening for one or, where appropriate, more than one chamber disposed side by side. The dispenser can be used in the vertical or horizontal position.

### BRIEF DESCRIPTION OF THE DRAWINGS

The invention is explained hereinafter by means of an exemplary embodiment. The relevant drawing shows:

FIG. 1: a section through a portion of a storage device according to the invention; and

FIG. 2: a perspective view of the storage device.

### DETAILED DESCRIPTION OF THE PREFERRED EMBODIMENT

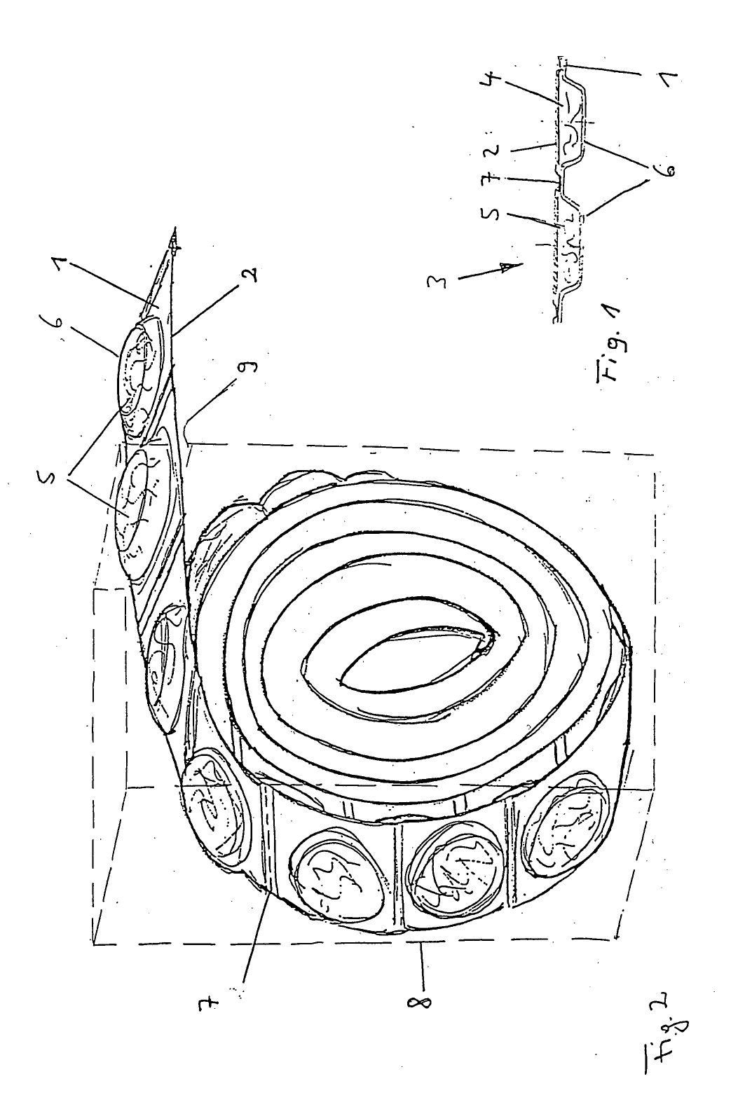

The storage device depicted in section in FIG. 1 has an envelope 3 which consists of a flexible base layer 1 made of polyamide/polyethylene and a flexible covering layer 2 (made of sterilization kraft paper), and which is provided with chambers 4 each for one swab 5. The chambers 4 are formed by the receiving depressions 6 provided in the base layer 1 and by the covering layer 2 disposed on top of these, and are sealed by this base layer 1 in the areas between the receiving depressions 6. This means that the swabs 5 kept in stock in the chambers 4 are sealed bacteria-tight. The welding points are provided simultaneously as strips or points 7 intended to be torn through, with reduced thickness of the covering layer. The chambers 4 are disposed one after the other in a row.

FIG. 2 shows the storage device with envelope 3 rolled up. A dispenser 8 is indicated by dashed lines, and it surrounds the rolled-up envelope 3 and has a removal opening 9 through which the envelope 3 can be pulled and divided into single chambers at the strips 7 intended to be torn through. The chambers 4 are opened by pressing a finger on the covering layer, causing it to tear inwards.

The invention has been described with particular emphasis on the preferred embodiments. It should be appreciated that these embodiments are described for purposes of illustration only, and that numerous alterations and modifications may be practiced by those skilled in the art without departing from the spirit and scope of the invention. It is intended that all such modifications and alterations be included insofar as they come within the scope of the invention or the equivalents thereof.

We claim:

1. A method for storing medical swabs comprising:
keeping medical swabs in an envelope comprising a flexible base layer and a flexible cover layer having adjacently disposed chambers between said layers, the size of said chambers being determined by the number and size of swabs to be stored, wherein the flexible base layer and the flexible cover layer are integrally bonded in areas between the chambers, and the envelope being sufficiently flexible enabling the envelope to be kept in stock rolled up or concertinaed.

2. Method according to claim 1, wherein the chambers comprise receiving depressions in the flexible base layer.

3. Method according to claim 2, wherein the receiving depressions are convex with respect to the flexible cover layer.

4. Method according to claim 1, wherein said flexible base layer is made of material selected from the group consisting of polyamide and polyethylene, and wherein said flexible covering layer consists of sterilization paper.

5. Method according to claim 1, wherein the chamber may be broken in response to finger pressure on the flexible cover layer to provide access to the chambers holding the medical swabs.

6. Method for storing medical swabs comprising:
determining the number and size of swabs to be stored in a chamber;
forming adjacently disposed chambers in a flexible base layer, the size of a chamber being determined by the number and size of swabs to be stored in said chamber;
placing the swabs to be stored in the chambers;
integrally bonding a flexible cover layer to the base layer in areas between the chambers, forming an envelope;
rolling up or concertinaeing said envelope for storage of the medical swabs.

7. Method according to claim 6, wherein the step of forming adjacently disposed chambers comprises forming receiving depressions in the flexible base layer to form said chambers.

8. Method according to claim 7, wherein the step of forming adjacently disposed chambers comprises forming convex receiving depressions in the flexible base layer with respect to the flexible cover layer.

9. Method according to claim 6, wherein said flexible base layer is made of material selected from the group consisting of polyamide and polyethylene, and wherein said flexible covering layer consists of sterilization paper.

10. Method according to claim 6, and further including the method for opening the chamber, said method comprises the step of:
breaking the chamber in response to finger pressure on the flexible cover layer to provide access to the medical swabs.
